(19) United States
(12) Patent Application Publication
(10) Pub. No.: US 2020/0144862 A1
(43) Pub. Date: May 7, 2020

(54) CHARGING SYSTEM, CHARGING DEVICE, AND CONTROL DEVICE OF ELECTRICAL TREATMENT DEVICE

(71) Applicant: OMRON HEALTHCARE Co., Ltd., Kyoto (JP)

(72) Inventor: Kenichiro ZAITSU, Kyoto (JP)

(21) Appl. No.: 16/736,388

(22) Filed: Jan. 7, 2020

Related U.S. Application Data
(63) Continuation of application No. PCT/JP2018/022372, filed on Jun. 12, 2018.

(30) Foreign Application Priority Data
Jul. 28, 2017 (JP) 2017-146588

(51) Int. Cl. H02J 50/10; H02J 50/80; H02J 7/02; H02J 7/00; A61N 1/36

(52) U.S. Cl. CPC H02J 50/10; H02J 50/80; A61N 1/36014; H02J 7/0047; H02J 7/00045; H02J 7/02

(57) ABSTRACT

A charging system includes a control device (4) of an electrical treatment device and a charging device (6) that charges the control device (4) by contactless charging. The charging device (6) includes a support portion and a holding portion with a recessed shape formed in the support portion for fitting the control device (4) into. The control device (4) includes a light emission unit (406) that emits light when the control device (4) is fit in the holding portion. The charging device (6) includes a light reception unit (450) that receives light from the light emission unit (406), an authentication unit (452) that executes authentication processing in which first information based on light received and predetermined second information are compared; a transmitter coil (72) for transmitting power; and a power transmission control unit (456) that transmits power from the transmitter coil when authentication by the authentication unit is successful. The control device (4) includes a receiver coil (54) that receives power from the transmitter coil (72) and a rechargeable battery (240) chargeable by power received by the receiver coil (54).

### CROSS-REFERENCE TO RELATED APPLICATIONS

This is a continuation of International Application PCT/JP2018/022372, with an international filing date of Jun. 12, 2018, filed by applicant, the disclosure of which is hereby incorporated by reference in its entirety.

### TECHNICAL FIELD

The present disclosure relates to technology for contactless charging of a control device of an electrical treatment device.

### BACKGROUND ART

Contactless charging systems for contactless (wireless) charging of small electronic devices such as mobile phones have appeared in recent years. Using a contactless charging system to charge a rechargeable battery in a small electronic device greatly reduces the complexity of the charging operation.

For example, JP 2015-507468 T (Patent Document 1) describes a method and a device for wirelessly charging a portable electronic device such as a mobile device.

### CITATION LIST

### Patent Literature

Patent Document 1: JP 2015-507468 T

### SUMMARY OF INVENTION

### Technical Problem

An electrical treatment device (for example, a low-frequency treatment device) that performs treatment such as easing shoulder stiffness includes a control device that controls various processes as a main body portion, removable pads, and the like. The control device includes a rechargeable battery as a power source and is typically a relatively small device. Thus, there is a demand to reduce the complexity of the charging operation by charging the rechargeable battery by contactless charging. There is also a growing demand to prevent devices other than the intended control device, such as imitations of the control device, from being charged.

An object of an embodiment of the present disclosure is to provide a charging system capable of easily and appropriately charging, by contactless charging, a control device of an electrical treatment device held in a charging device. Also, an object of another embodiment of the present disclosure is to provide a charging device and a control device of an electrical treatment device used in such a charging system.

### Solution to Problem

According to an embodiment, a charging system includes a control device of an electrical treatment device and a charging device that charges the control device by contactless charging. The charging device includes a support portion, and a holding portion formed in the support portion, the holding portion having a recessed shape for fitting the control device into. The control device includes a light emission unit that emits light when the control device is fit in the holding portion. The charging device further includes a light reception unit that receives light from the light emission unit, an authentication unit configured to execute authentication processing in which first information based on the light received and predetermined second information are compared, a transmitter coil for transmitting power, and a power transmission control unit configured to transmit power from the transmitter coil when authentication by the authentication unit is successful. The control device further includes a receiver coil that receives power from the transmitter coil, and a rechargeable battery chargeable by power received by the receiver coil.

Preferably, the holding portion includes a flat portion corresponding to a shape of a bottom surface of the control device and a curved surface portion corresponding to a curved surface shape formed from an outer edge of the bottom surface of the control device.

Preferably, the bottom surface of the control device is provided with a positioning recess portion, and the flat portion of the holding portion is provided with a positioning protrusion portion that interlocks with the positioning recess portion.

Preferably, the light emission unit emits light at predetermined intervals when the control device is fit in the holding portion. Also, the authentication unit is configured to execute the authentication processing each time light is received by the light reception unit.

Preferably, the power transmission control unit is configured to cause power to stop being transmitted from the transmitter coil when power is transmitted from the transmitter coil and authentication by the authentication unit fails.

Preferably, the light emission unit is configured to stop emitting light when charging of the rechargeable battery is complete.

Preferably, the electrical treatment device is a low-frequency treatment device.

According to another embodiment, a charging device for charging a control device of an electrical treatment device by contactless charging is provided. The charging device includes a support portion, a holding portion formed in the support portion, the holding portion having a recessed shape for fitting the control device into, a light reception unit that receives light from the control device when the control device is fit in the holding portion, an authentication unit configured to execute authentication processing in which first information based on the light received and predetermined second information are compared, a transmitter coil for transmitting power, and a power transmission control unit configured to transmit power from the transmitter coil to a receiver coil of the control device when authentication by the authentication unit is successful.

According to yet another embodiment, a control device of an electrical treatment device chargeable by contactless charging by power from a charging device is provided. The control device includes a light emission unit that emits light when the control device is fit in a holding portion having a recessed shape formed in a support portion of the charging device, a receiver coil that receives power from a transmitter coil of the charging device when authentication processing is successful, the authentication processing including comparing first information based on light received by a light reception unit of the charging device and predetermined second information, and a rechargeable battery chargeable by power received by the receiver coil.

### Advantageous Effects of Invention

According to the present disclosure, a control device of an electrical treatment device held in a charging device can be easily and appropriately charged by contactless charging.

### BRIEF DESCRIPTION OF DRAWINGS

FIG. 1 is a perspective view illustrating a charging system according to the present embodiment, with a control device and a charging device separated from one another.

FIG. 2 is a perspective view illustrating the charging system according to the present embodiment, with the control device set in the charging device.

FIG. 3 is a plan view illustrating a pad and a holder of an electrical treatment device according to the present embodiment.

FIG. 4 is a perspective view illustrating the control device of the electrical treatment device according to the present embodiment.

FIG. 5 is a plan view illustrating the control device of the electrical treatment device according to the present embodiment.

FIG. 6 is a side view as seen in the direction of arrow VI in FIG. 4.

FIG. 7 is a bottom view illustrating the control device of the electrical treatment device according to the present embodiment.

FIG. 8 is a perspective view illustrating the charging device according to the present embodiment.

FIG. 9 is a plan view illustrating the charging device according to the present embodiment.

FIG. 10 is a block diagram illustrating an example of the hardware configuration of the control device of the electrical treatment device according to the present embodiment.

FIG. 11 is a block diagram illustrating an example of the hardware configuration of the charging device according to the present embodiment.

FIG. 12 is a block diagram illustrating the functional configuration of the control device and the charging device according to the present embodiment.

FIG. 13 is a flowchart illustrating an example of the processing procedure of the control device and the charging device according to the present embodiment.

### DESCRIPTION OF EMBODIMENTS

Embodiments of the present invention will be described below with reference to the drawings. In the following description, like components are given like numerals. Names and functions thereof are also the same. Thus, the detailed description of such components is not repeated.

General Configuration

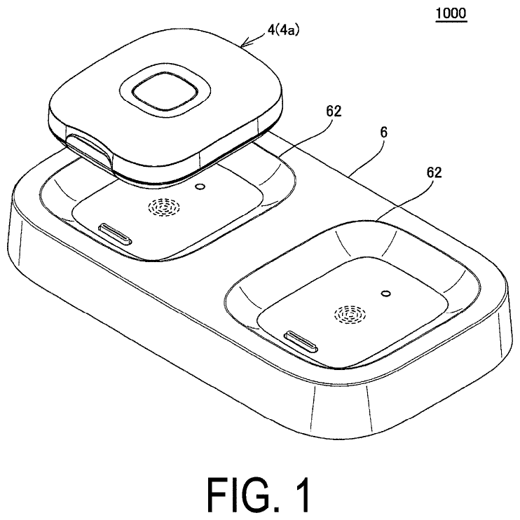

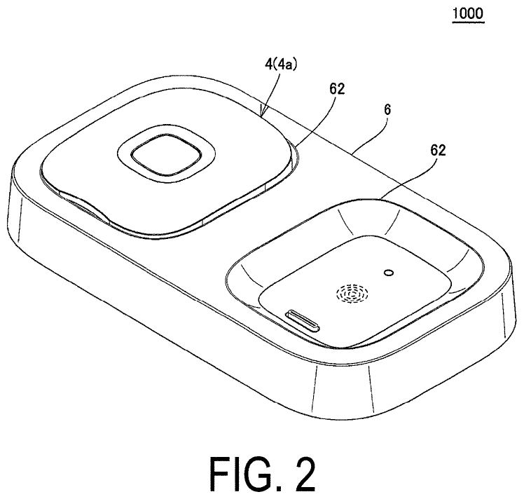

The general configuration of a charging system according to the present embodiment will be described. FIG. 1 is a perspective view illustrating the charging system according to the present embodiment, with a control device and a charging device separated from one another. FIG. 2 is a perspective view illustrating the charging system according to the present embodiment, with the control device set in the charging device.

Referring to FIGS. 1 and 2, a charging system 1000 includes a control device 4 (case 4a), which is a main body portion of an electrical treatment device, and a charging device 6 for charging the control device 4. The electrical treatment device according to the present embodiment is a so-called cordless type low-frequency treatment device.

The control device 4 includes a rechargeable battery. As illustrated in FIG. 2, when charging the rechargeable battery, the control device 4 (case 4a) is fit and held in a holding portion 62 of the charging device 6. The holding portion 62 has a recessed shape. Specifically, a lower member of the case 4a and the holding portion 62 of the charging device 6 engage with one another, and the case 4a and the charging device 6 are held together with their opposing surfaces in close contact.

The rechargeable battery in the control device 4 is charged by contactless charging (wireless charging). Contactless charging is performed by power being transmitted in a non-contact manner via electromagnetic induction between two coils, one coil being provided in the charging device 6 on the power transmitting side and the other being provided in the control device 4 on the power receiving side.

Thus, no member (for example, a metal terminal) is required for electrically connecting the control device 4 and the charging device 6 when charging the rechargeable battery. Also, the control device 4 is easily set in the charging device 6, and dust, moisture, and the like are prevented from entering the members described above and causing a short circuit.

### Configuration of Electrical Treatment Device

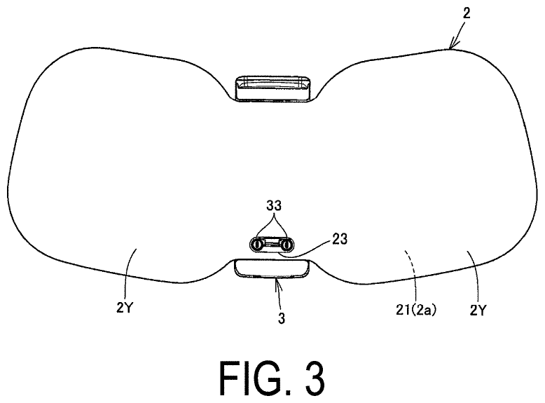

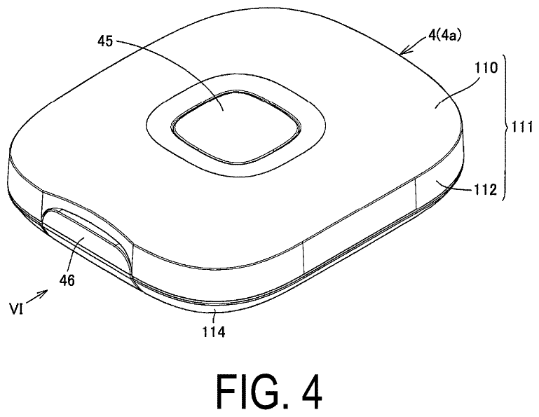

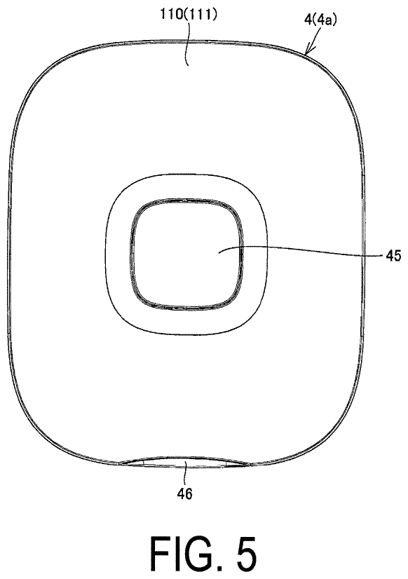

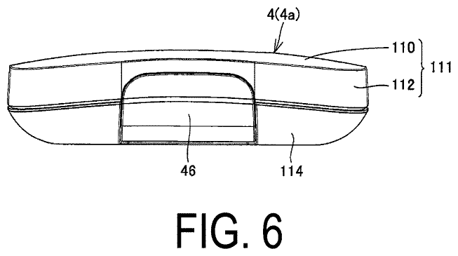

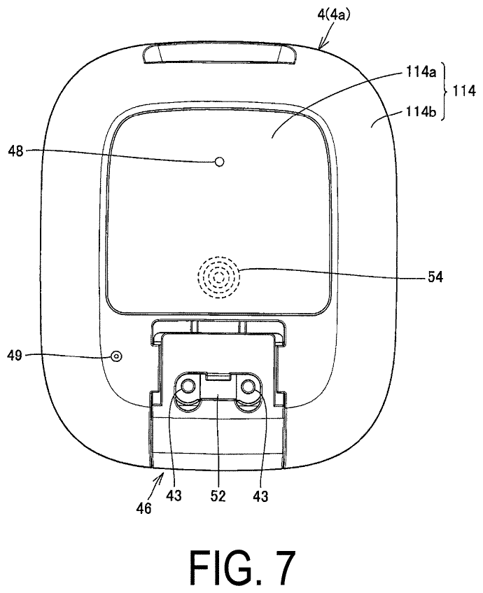

FIG. 3 is a plan view illustrating a pad and a holder of the electrical treatment device according to the present embodiment. FIG. 4 is a perspective view illustrating the control device of the electrical treatment device according to the present embodiment. FIG. 5 is a plan view illustrating the control device of the electrical treatment device according to the present embodiment. FIG. 6 is a side view as seen in the direction of arrow VI in FIG. 4. FIG. 7 is a bottom view illustrating the control device of the electrical treatment device according to the present embodiment.

The electrical treatment device according to the present embodiment includes a pad 2 and a holder 3 illustrated in FIG. 3 and the control device 4, which is the main body portion, illustrated in FIGS. 4 to 7.

Referring to FIG. 3, the pad 2 has a sheet-like shape and is configured to attach to the user's body. A conductive layer 2a is provided on a body-side portion 21 surface (lower surface), of the outer surfaces of the pad 2, that faces the body. The pad 2 is attached to the user's skin by using a conductive gel or the like, and a low-frequency pulse is supplied to the user through the conductive layer 2a.

The pad 2 is held by the holder 3. A through hole 23 is provided in the pad 2, and a protrusion-shaped terminal 33 of the holder 3 is insertable in the through hole 23. A treatment portion 2Y of the pad 2 is provided on both the left and right sides, and a conductive layer 2a is exposed at a body-side portion 21 of the treatment portion 2Y.

The protrusion-shaped terminal 33 of the holder 3 is formed for electrical connection with a terminal hole 43 (see FIG. 7) on the control device 4. When the protrusion-shaped terminal 33 is inserted into the terminal hole 43 on the control device 4 (when connected), a pulse current is supplied to the user via the conductive layer 2a of the pad 2.

The pad 2 is a consumable item, and the pad 2 can be detachably attached to the control device 4, thus allowing replacement of the pad 2. In the present embodiment, the holder 3 holds the pad 2 such that the holder 3 and the pad 2 are integrated, and the control device 4 is configured to be detachably attached to the pad 2 and the holder 3. The pad 2 can be replaced together with the holder 3, or it is also possible to reuse the holder 3 as necessary.

Referring to FIGS. 4 to 7, the control device 4 includes as an outer cover the case 4a with a substantially rectangular parallelepiped shape. The control device 4 (case 4a) is detachably attached to the holder 3. With the control device 4 attached to the holder 3, the main body portion 4 supplies a low-frequency pulse current to the conductive layer 2a of the pad 2.

As illustrated in FIGS. 4 to 7, the case 4a includes an upper member 111 including an upper surface portion 110 and a side surface portion 112 and a lower member 114. The upper member 111 and the lower member 114 are connected. The upper surface portion 110 has a substantially rectangular shape and slightly curves as it extends toward to outer portions of the case 4a. A power button 45 is integrally formed in the upper surface portion 110. The side surface portion 112 has a substantially annular shape. A button 46 for attaching and detaching the holder 3 from the case 4a is provided on the side surface portion 112.

The lower member 114 includes a flat bottom surface portion 114a with a substantially rectangular shape and a curved surface portion 114b formed from the edge portion of the bottom surface portion 114a with a convex curved surface. The curved surface portion 114b with the upper surface portion 110 and the side surface portion 112 forms the three-dimensional shape of the control device 4.

The control device 4 (case 4a) is internally provided with a substrate, an electric circuit, a rechargeable battery, a receiver coil 54, and the like. The electric circuit is mounted on the surface of the substrate. Examples of the electric circuit include a processor for performing various processes, a memory for storing programs and data, a communication interface for wirelessly communicating various types of data with an external device, and a waveform generation output device for boosting the power source voltage and generating and outputting a low-frequency pulse current (treatment current).

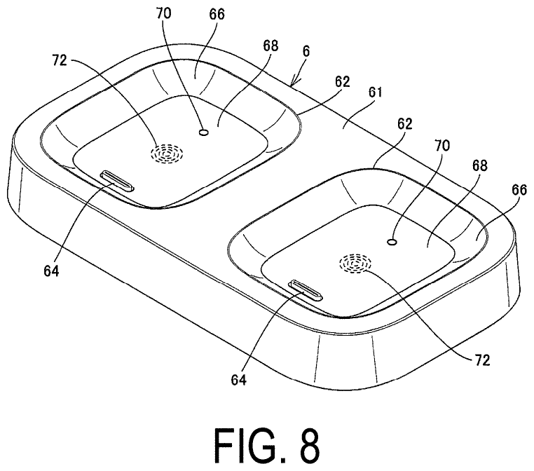

When current flows to a transmitter coil provided in the charging device 6 (a transmitter coil 72 illustrated in FIG. 8 described below), with the transmitter coil and the receiver coil 54 opposing one another, power is transmitted from the transmitter coil to the receiver coil 54 via electromagnetic induction. The control device 4 can utilize the power from the receiver coil 54 to charge the rechargeable battery.

A positioning recess portion 52 for positioning the control device 4 in relation to the holding portion 62 of the charging device 6 is disposed in the bottom surface portion 114a. By the positioning recess portion 52 being fit on a positioning protrusion portion provided on the holding portion 62, the control device 4 is held in the holding portion 62. In addition, the terminal hole 43 into which the protrusion-shaped terminal 33 of the holder 3 is inserted is internally provided in the positioning recess portion 52. The terminal hole 43 is electrically connected with the substrate (electric circuit) inside the case 4a.

The bottom surface portion 114a is provided with an LED 48, which is a light emission portion, and a reset button 49. The LED 48 emits light in accordance with an instruction of the processor of the control device 4. The reset button 49 is a button for initializing an electrical treatment device 20. For example, in response to receipt of a downward push operation of the reset button 49, the electrical treatment device 20 deletes the pairing information stored in the internal memory when a pairing connection was established with an external device.

### Charging Device Configuration

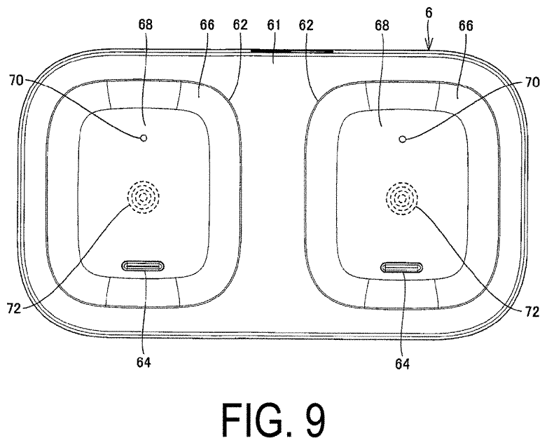

FIG. 8 is a perspective view illustrating the charging device 6 according to the present embodiment. FIG. 9 is a plan view illustrating the charging device 6 according to the present embodiment. The charging device 6 holds the control device 4 and charges the rechargeable battery accommodated in the control device 4. Referring to FIGS. 8 and 9, the charging device 6 includes a support portion 61 and two holding portions 62 formed in the surface of the support portion 61.

The support unit 61 is a base that supports the control device 4 (case 4a) held by the holding portion 62. The holding portions 62 are formed in the substantially rectangular upper surface of the support portion 61. In the present embodiment, two holding portions 62 are formed in the support portion 61. Thus, two control devices 4 can be charged at the same time by fitting the control devices 4 into the holding portions 62. However, in another embodiment, one holding portion 62 may be provided in the support portion 61.

The holding portion 62 represents the portion that holds the control device 4 and is formed with a recess shaped conforming to the shape of the lower member 114 of the case 4a. Specifically, the holding portion 62 has a recessed shape for fitting the lower member 114 into. In addition, as illustrated in FIG. 9, the outer edge of the holding portion 62 as seen from the front has a substantially rectangular shape.

The holding portion 62 includes a flat portion 68 and a curved surface portion 66. The flat portion 68 opposes the bottom surface portion 114a of the lower member 114 held in the holding portion 62. The curved surface portion 66 is a concave curved surface and opposes the curved surface portion 114b of the lower member 114 held in the holding portion 62. In other words, the holding portion 62 includes the flat portion 68 that corresponds to the shape of the bottom surface portion 114a of the control device 4 and the curved surface portion 66 that corresponds to the curved surface portion 114b formed from the outer edge of the bottom surface portion 114a.

A positioning protrusion portion 64 that interlocks with the positioning recess portion 52 of the control device 4 (case 4a) is disposed on the flat portion 68 of the holding portion 62. The case 4a is held by the inner peripheral edge of the positioning recess portion 52 being fit into the positioning protrusion portion 64.

A light receiving element 70 is a photodetector such as a photodiode. With the control device 4 held in the holding portion 62, the light receiving element 70 is disposed opposing the LED 48 provided in the lower member 114 of the case 4a.

The transmitter coil 72 for transmitting power to the receiver coil 54, a power source connection terminal that connects to an external power source, and the like are internally provided in the support portion 61 of the charging device 6. With the control device 4 (case 4a) held in the holding portion 62, the transmitter coil 72 is disposed opposing the receiver coil 54 of the control device 4. When current is input from a connected external power source via a power source connection terminal, the current flows to the transmitter coil 72 and a magnetic field is generated. A current is induced in the receiver coil 54 of the control device 4 by the magnetic flux.

A substrate (not illustrated) and an electric circuit (not illustrated) are also internally provided in the support portion 61. The electric circuit includes a processor that receives an input of an electrical signal converted by the light receiving element 70 and executes various processes, memory, and the like.

### Hardware Configuration

### Control Device of Electrical Treatment Device

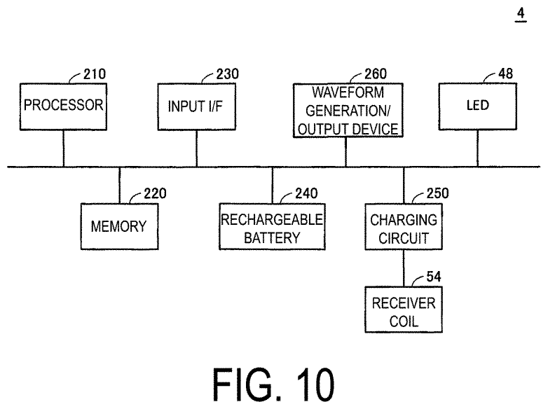

FIG. 10 is a block diagram illustrating an example of the hardware configuration of the control device of the electrical treatment device according to the present embodiment. Referring to FIG. 10, the control device 4 includes, as main components, a processor 210, a memory 220, an input interface (I/F) 230, a rechargeable battery 240, a charging circuit 250, a waveform generation/output device 260, the receiver coil 54, and the LED 48.

The processor 210 typically may be an arithmetic processing unit such as a central processing unit (CPU) or a multi processing unit (MPU). The processor 210 functions as a control unit that controls the operation of components of the control device 4 by reading out and executing a program stored in the memory 220. By executing the program, the processor 210 executes processing (steps) of the control device 4 described later.

The memory 220 is realized by random access memory (RAM), read-only memory (ROM), and the like. The memory 220 stores programs executed by the processor 210, data used by the processor 210, and the like. The input interface 230 includes a variety of buttons for receiving instructions from a user.

The rechargeable battery 240 supplies power to the components of the control device 4. The rechargeable battery 240 is, for example, a lithium ion battery. The charging circuit 250 is connected to the receiver coil 54 for charging and uses power from the receiver coil 54 to charge the rechargeable battery 240.

The waveform generation/output device 260 outputs a current that flows to the treatment site of the user's body via the pad 2. The waveform generation/output device 260 includes a booster circuit, a voltage adjustment circuit, an output circuit, a current detection circuit, and the like.

### Charging Device

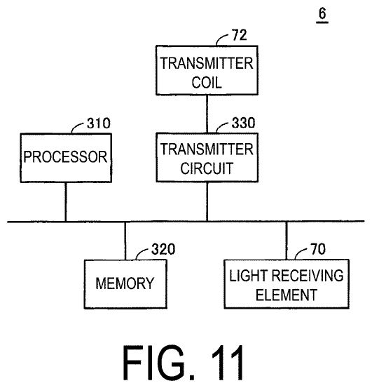

FIG. 11 is a block diagram illustrating an example of the hardware configuration of the charging device according to the present embodiment. Referring to FIG. 11, the charging device 6 includes, as main components, a processor 310 for executing various processes, a memory 320 that stores various types of information, a transmitter circuit 330, the transmitter coil 72, and the light receiving element 70.

The light receiving element 70 converts received light into an electrical signal and inputs the electrical signal to the processor 310. The processor 310 controls the transmitter circuit 330 on the basis of the electrical signal converted by the light receiving element 70. The transmitter circuit 330 supplies to the transmitter coil 72 AC power from an external power source such as a home power source in accordance with an instruction of the processor 310.

### Functional Configuration

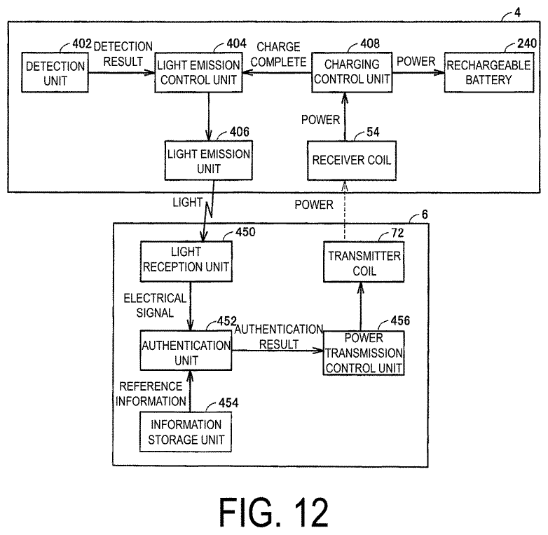

FIG. 12 is a block diagram illustrating the functional configuration of the control device 4 and the charging device 6 according to the present embodiment. Referring to FIG. 12, the control device 4 includes, as a main functional configuration, a detection unit 402, a light emission control unit 404, a light emission unit 406, and a charging control unit 408. The charging device 6 includes, as a main functional configuration, a light reception unit 450, an authentication unit 452, an information storage unit 454, and a power transmission control unit 456.

The detection unit 402 of the control device 4 detects the holding state of the control device 4 with respect to the holding portion 62 of the charging device 6. Specifically, the detection unit 402 detects whether the positioning recess portion 52 of the control device 4 is fitted on the positioning protrusion portion 64 provided on the holding portion 62 of the charging device 6 (i.e., the control device 4 is in a held state) or whether it is not fitted (i.e., the control device 4 is in a non-held state).

For example, the detection unit 402 may mechanically detect that the positioning protrusion portion 64 is fitted into the positioning recess portion 52 via a switch (not illustrated) provided on the positioning recess portion 52. Also, the detection unit 402 may detect that the positioning protrusion portion 64 is fitted into the positioning recess portion 52 on the basis of a change in voltage when the positioning protrusion portion 64 comes into contact with a terminal (not illustrated) provided on the positioning recess portion 52.

Information of the detection results obtained by the detection unit 402 (for example, information indicating that the control device 4 is held in the holding portion 62) is supplied to the light emission control unit 404.

When the control device 4 is fitted into the holding unit 62, the light emitting control unit 404 causes light to be emitted from the light emission unit 406. Light from the light emission unit 406 is used in authentication processing of the charging device 6. Specifically, when the control device 4 is fitted into the holding unit 62, the light emission control unit 404 causes light to be emitted from the light emission unit 406 at predetermined intervals (for example, every 1 second). When the control device 4 is released from its held state in the holding unit 62, the light emitting control unit 404 causes the light emission unit 406 to stop emitting light. The light emission control unit 404 is realized by the processor 210, for example. The light emission unit 406 is realized by the LED 48, for example.

The light reception unit 450 of the charging device 6 receives light from the light emission unit 406, converts the light into an electrical signal, and supplies the electrical signal to the authentication unit 452. The light reception unit 450 is realized by the light receiving element 70, for example.

The authentication unit 452 authenticates whether the light received by the light reception unit 450 indicates allowing power to be transmitted to the control device 4. Specifically, the authentication unit 452 executes authentication processing in which information based on the received light and predetermined reference information are compared. The reference information is information indicating the definition of light that allows power to be transmitted, and is stored in the information storage unit 454 realized by the memory 320. For example, the reference information is information relating to the light emitted by the LED 48 of the control device 4. The information relating to light may be information indicating wavelength characteristics of light or may be information indicating transmission strength of a specific wavelength.

For example, the authentication unit 452 may compare the wavelength characteristics of the received light and the wavelength characteristics indicated by the reference information and determine that authentication is successful in a case where the two match. Also, the authentication unit 452 may compare the transmission strength of the specific wavelength of the received light and the transmission strength of the specific wavelength indicated by the reference information and determine that authentication is successful in a case where the two match. In other words, the authentication unit 452 determines that authentication is successful if the received light is confirmed to be the light emitted by the LED 48 in the control device 4.

The authentication unit 452 executes authentication processing each time light is received by the light reception unit 450 (each time light is emitted from the light emission unit 406 at predetermined intervals). The authentication result of the authentication unit 452 is supplied to the power transmission control unit 456. The authentication unit 452 is realized by the processor 310, for example.

When authentication via the authentication unit 452 is successful, the power transmission control unit 456 causes power to be transmitted from the transmitter coil 72. In another embodiment, when power is being transmitted from the transmitter coil 72 and authentication via the authentication unit 452 fails, the power transmission control unit 456 causes power to stop being transmitted from the transmitter coil 72. The power transmission control unit 456 is realized by the processor 310 and the transmitter circuit 330, for example.

The charging control unit 408 of the control device 4 receives power from the transmitter coil 72 via the receiver coil 54 and supplies the power to the rechargeable battery 240. Also, the charging control unit 408 is configured to detect the remaining battery power of the rechargeable battery 240. The output voltage and the output current of the rechargeable battery decreases depending on the remaining battery power. Thus, the charging control unit 408 detects the remaining battery power on the basis of the output voltage and/or the output current. When charging of the rechargeable battery 240 is complete (i.e., the remaining battery power is 100% and at full charge state), the charging control unit 408 supplies information indicating charge completion to the light emission control unit 404. When charging of the rechargeable battery 240 is complete, the light emission control unit 404 causes light to be emitted by the light emission unit 406.

### Processing Procedure

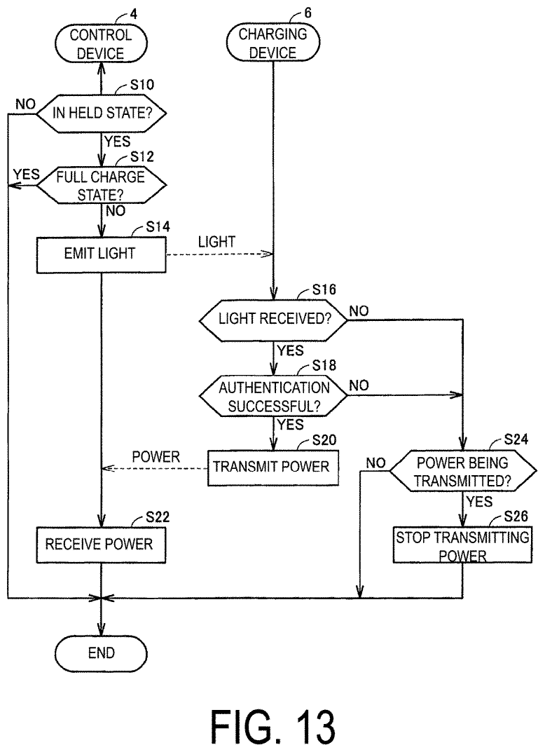

FIG. 13 is a flowchart illustrating an example of the processing procedure of the control device and the charging device according to the present embodiment. For example, the steps of the control device 4 described below are executed by the processor 210, and the steps of the charging device 6 are executed by the processor 310. The following processes are repeated at predetermined intervals (control cycles).

Referring to FIG. 13, the control device 4 determines whether the case 4a is held in the holding portion 62 of the charging device 6 (step S10). Specifically, the control device 4 determines whether the positioning recess portion 52 and the positioning protrusion portion 64 are fitted together.

If the case 4a is held in the holding portion 62 (YES in step S10), the control device 4 determines whether the rechargeable battery 240 is in a full charge state (i.e., charge complete state) (step S12). If the rechargeable battery 240 is not in a full charge state (NO in step S12), the control device 4 causes light to be emitted from the LED 48 (step S14).

If the case 4a is not held in the holding portion 62 (NO in step S10), the control device 4 ends the process. In this case, the LED 48 does not emit light. If the rechargeable battery 240 is in a full charge state (YES in step S12), the control device 4 ends the process. In this case also, the LED 48 does not emit light. That is, when the case 4a is held in the holding portion 62 and the rechargeable battery 240 is in a full charge state, the control device 4 does not cause the LED 48 to emit light (stops light emitting from the LED 48).

The charging device 6 determines whether light has been received via the light receiving element 70 (step S16). If light has not been received (NO in step S16), the charging device 6 executes the process of step S24 described below. If light has been received (YES in step S16), the charging device 6 determines whether authentication processing was successful on the basis of the received light (step S18). Specifically, the charging device 6 executes authentication processing for allowing power to be transmitted to the control device 4 by comparing information on the basis of the received light and the reference information and then performs the determination.

If authentication is successful (YES in step S18), the charging device 6 transmits power from the transmitter coil 72 (step S20). When power is being transmitted from the transmitter coil 72, the power transmission is maintained. The control device 4 receives power via the receiver coil 54 (step S22) and ends the process.

If authentication is failed (NO in step S18), the charging device 6 determines whether power is being transmitted from the transmitter coil 72 (step S24). If power is being transmitted (YES in step S24), the charging device 6 stops power being transmitted from the transmitter coil 72 (step S26) and ends the process. If power is not being transmitted (NO in step S24), the charging device 6 ends the process. In this case, the transmitter coil 72 does not start transmitting power.

### Advantages

According to the present embodiment, with the control device 4 held in the holding portion 62, unwanted foreign light reaching the light receiving element 70 is blocked and the LED 48 and the light receiving element 70 are disposed opposing one another. With the light receiving element 70, the detection sensitivity of light from the LED 48 is enhanced, and authentication processing with high accuracy can be executed.

According to the present embodiment, charging automatically starts when the control device 4 is fit in the holding portion 62, thus enhancing user convenience.

According to the present embodiment, the authentication processing is executed at predetermined intervals, and power transmission is stopped if the authentication fails. In this way, security is also enhanced by constantly executing authentication.

### Other Embodiments

(1) In the embodiment described above, the control device 4 of the electrical treatment device may be configured to not communicate with an external device or the like or not output a treatment current when the charging device 6 is held in the holding portion 62.

(2) In the embodiment described, the arrangement of the LED 48 and the light receiving element 70 is not limited to that described above and it is only required that, with the control device 4 held in the holding portion 62, the light receiving element 70 and the LED 48 be disposed opposing one another.

(3) In the embodiments described above, a program may be provided that causes a computer to function and execute controls such as those described in the flowcharts described above. Such a program can also be provided as a program product stored on a non-temporary computer-readable recording medium attached to a computer, such as a flexible disk, a compact disk read only memory (CD), a secondary storage device, a main storage device, and a memory card. Alternatively, a program may be provided, which is stored on a recording medium such as a hard disk built into a computer. The program may also be provided by download via a network.

With the program, required modules from among program modules provided as part of the computer operating system (OS) may be called in a predetermined sequence at a predetermined timing to execute processing. In this case, the modules described above are not included in the program itself, and the process is executed in cooperation with the OS. Programs that do not include such modules may also be included in the program according to the present embodiment.

In addition, the program according to the present embodiment may be provided integrated into a part of a different program. In this case as well, the program according to the present embodiment per se does not include the modules included in the different program described above, and the process is executed in cooperation with the different programs. Such a program integrated in a different program shall also be within the scope of the program according to the present embodiment.

(4) The configuration given as an example of the embodiment described above is an example configuration of the present invention. The configuration can be combined with other known technology, and parts thereof may be omitted or modified within the scope of the present invention. Furthermore, the processes and configurations of other embodiments may be employed as appropriate to the embodiments described above.

The embodiments described herein are illustrative in all respects and are not intended as limitations. The scope of the present invention is indicated not by the descriptions above but by the claims and includes all meaning equivalent to the scope and changes within the scope.

### REFERENCE SIGNS LIST

2 Pad
2Y Treatment portion
2a Conductive layer
3 Holder
4 Control device
4a Case
6 Charging device
20 Electrical treatment device
21 Body-side portion
23 Through hole
33 Protrusion-shaped terminal
43 Terminal hole
45 Power button
46 Button
49 Reset button
52 Positioning recess portion
54 Receiver coil
61 Support portion
62 Holding portion
64 Positioning protrusion portion
66, 114b Curved surface portion
68 Flat portion
70 Light receiving element
72 Transmitter coil
110 Upper surface portion
111 Upper member
112 Side surface portion
114 Lower member
114a Bottom surface portion
210, 310 Processor
220, 320 Memory
230 Input interface
240 Rechargeable battery
250 Charging circuit
260 Waveform generation/output device
330 Transmitter circuit
402 Detection unit
404 Light emission control unit
406 Light emission unit
408 Charging control unit
450 Light reception unit
452 Authentication unit
454 Information storage unit
456 Power transmission control unit
1000 Charging system

1. A charging system, comprising:
a control device of an electrical treatment device; and
a charging device that charges the control device by contactless charging; wherein
the charging device comprises
a support portion, and
a holding portion formed in the support portion, the holding portion having a recessed shape for fitting the control device into;
the control device comprises
a light emission unit that emits light when the control device is fit in the holding portion;
the charging device further comprises
a light reception unit that receives light from the light emission unit,
an authentication unit configured to execute authentication processing in which first information based on the light received and predetermined second information are compared,
a transmitter coil for transmitting power, and
a power transmission control unit configured to transmit power from the transmitter coil when authentication by the authentication unit is successful; and
the control device further comprises
a receiver coil that receives power from the transmitter coil, and
a rechargeable battery chargeable by power received by the receiver coil.

2. The charging system according to claim 1, wherein
the holding portion comprises a flat portion corresponding to a shape of a bottom surface of the control device and a curved surface portion corresponding to a curved surface shape formed from an outer edge of the bottom surface of the control device.

3. The charging system according to claim 2, wherein
the bottom surface of the control device is provided with a positioning recess portion, and
the flat portion of the holding portion is provided with a positioning protrusion portion that interlocks with the positioning recess portion.

4. The charging system according to claim 1, wherein
the light emission unit emits light at predetermined intervals when the control device is fit in the holding portion, and
the authentication unit is configured to execute the authentication processing each time light is received by the light reception unit.

5. The charging system according to claim 4, wherein
the power transmission control unit is configured to cause power to stop being transmitted from the transmitter coil when power is transmitted from the transmitter coil and authentication by the authentication unit fails.

6. The charging system according to claim 1, wherein
the light emission unit is configured to stop emitting light when charging of the rechargeable battery is complete.

7. The charging system according to claim 1, wherein the electrical treatment device is a low-frequency treatment device.

8. A charging device for charging a control device of an electrical treatment device by contactless charging, the charging device comprising:
a support portion;
a holding portion formed in the support portion, the holding portion having a recessed shape for fitting the control device into;
a light reception unit that receives light from the control device when the control device is fit in the holding portion;
an authentication unit configured to execute authentication processing in which first information based on the light received and predetermined second information are compared;
a transmitter coil for transmitting power; and
a power transmission control unit configured to transmit power from the transmitter coil to a receiver coil of the control device when authentication by the authentication unit is successful.

9. A control device of an electrical treatment device chargeable by contactless charging by power from a charging device, the control device comprising:
a light emission unit that emits light when the control device is fit in a holding portion having a recessed shape formed in a support portion of the charging device;
a receiver coil that receives power from a transmitter coil of the charging device when authentication processing is successful, the authentication processing comprising comparing first information based on light received by a light reception unit of the charging device and predetermined second information; and
a rechargeable battery chargeable by power received by the receiver coil.
